(19) United States
(12) Patent Application Publication
(10) Pub. No.: US 2011/0182909 A1
(43) Pub. Date: Jul. 28, 2011

(54) Glufosfamide combination therapy

(75) Inventor: George Tidmarsh, Portola Valley, CA (US)

(73) Assignee: Threshold Pharmaceuticals, Inc., Redwood City, CA (US)

(21) Appl. No.: 11/794,110

(22) PCT Filed: Dec. 22, 2005
(86) PCT No.: PCT/US2005/047314

§ 371 Date: Nov. 27, 2007

Related U.S. Application Data
(60) Provisional application No. 60/638,995, filed on Dec. 23, 2004.

(51) Int. Cl. A61K 39/395; A61K 31/7024

(52) U.S. Cl. 424/155.1; 514/25

(57) ABSTRACT

Glufosfamide administered in combination with other chemotherapeutic agents is useful in cancer treatment.

### CROSS-REFERENCE TO RELATED APPLICATIONS

This application claims the benefit of U.S. Patent Application No. 60/638,995, filed Dec. 23, 2004, the contents of which are incorporated herein by reference.

### FIELD OF THE INVENTION

The present invention provides compositions and methods for treating cancer with glufosfamide in combination with anticancer agents, and generally relates to the fields of chemistry, biology, molecular biology, pharmacology, and medicine.

### BACKGROUND OF THE INVENTION

The lymphatic system is defined as the interconnected system of spaces and vessels between body tissues and organs by which lymph circulates throughout the body. The lymphatic system includes the bone marrow, spleen, thymus, tonsils, lymph, lymph nodes, lymphocytes, and lymphatic vessels. Lymphoma is the general term for primary cancer of the lymphatic system, such as tumors that arise in the lymph nodes or in other lymphoid tissue. Lymphoma is defined as the uncontrolled growth and spread of abnormal cells of the lymphatic system, and the formation of solid tumors with cells arising from proliferating lymphocytes. Symptoms of the disease include lymph node swelling, weight loss and fever. The most common type of lymphoma is Hodgkin's Disease (HD). The other more than twenty closely related lymphomas are collectively referred to as non-Hodgkin's Lymphoma (NHL).

Lymphoma currently accounts for approximately five percent of all cancers, with approximately 63,000 new cases expected to be diagnosed in 2004. Lymphoma is the most common blood cancer and the third most common cancer of childhood. NHL accounts for approximately 88% of all lymphomas diagnosed each year. Aside from melanoma of the skin, NHL is the only cancer whose incidence rate in the United States is on the rise.

Treatment of patients with lymphoma involves radiation therapy (radiotherapy), chemotherapy, or both, followed by second-line high dose chemotherapy and optionally some type of stem cell transplant (SCT) therapy. With current first-line therapies, only 50-60% of patients with aggressive NHL achieve a remission. An effective, high dose, second line chemotherapy is required for patients not responding to first-line therapies and in those having disease relapse after remission. A good partial response (PR) or complete response (CR) in the second line chemotherapy treatment is needed for subsequent successful SCT therapy. Furthermore, transplantation success is tightly correlated with overall cytoreduction of the tumor, and with the mobilization of peripheral stem cells (Moskowitz, et al., 1999, J Oncol. 17(12):3776-85).

Previous second-line chemotherapies, including dexamethasone/cisplatin/cytarabine (DHAP), etoposide/high-dose cytarabine/cisplatinum (ESHAP), and BNCU (bis-chloro-ethylnitrosurea)/etoposide/cytarabine/melphalan (mini-BEAM) have not proven to be very effective at stem cell mobilization, and have exhibited high levels of non-hematological toxicity (Zelenetz et al., 2003, Ann. Oncol., 14 (Suppl. 1):i 5-10 incorporated herein by reference).

A combination chemotherapy of ifosfamide, carboplatin, and etoposide (ICE), has been used for such second line therapy (See for example, Zelenetz et al., supra and Hertzberg et al., Ann Oncol. 2003, 14 (Suppl. 1):i 11-16, incorporated herein by reference). Addition of Rituximab, a chimeric anti-CD20 monoclonal antibody, to the ICE protocol in CD20-positive patients has also been used (RICE). The RICE protocol improves the rate of complete response compared to the ICE protocol (see Kewalramani et al, 2004, Blood, 103(10):3684-8 and Zelenetz et al. supra, each of which is incorporated herein by reference). However, patients undergoing ICE and RICE therapies suffer from severe toxic events with ifosfamide being a leading cause of toxicity.

Ifosfamide is a lipophilic prodrug and is generally administered over a period of 24 hours, requiring hospitalization of the patient. Upon hydroxylation by liver cytochrome P450, ifosfamide yields the anti-neoplastic alkylator A and the cytotoxic byproduct acrolein as shown below (Hardman et al., Goodman & Gilman's The Pharmacological Basis of Therapeutics, 2001, 10^{th} Edition, McGraw-Hill, New York, 1390-6, incorporated herein by reference). Acrolein is postulated to cause hemorrhagic cystitis, and the uroprotectant mesna is generally coadministered with ifosfamide. However mesna itself exhibits toxicity in patients (see, for example, Reinhold-Keller E, Clin Investig. 1992, 70(8):698-704). Additionally, the metabolism of ifosfamide also generates the severely toxic chloroacetaldehyde.

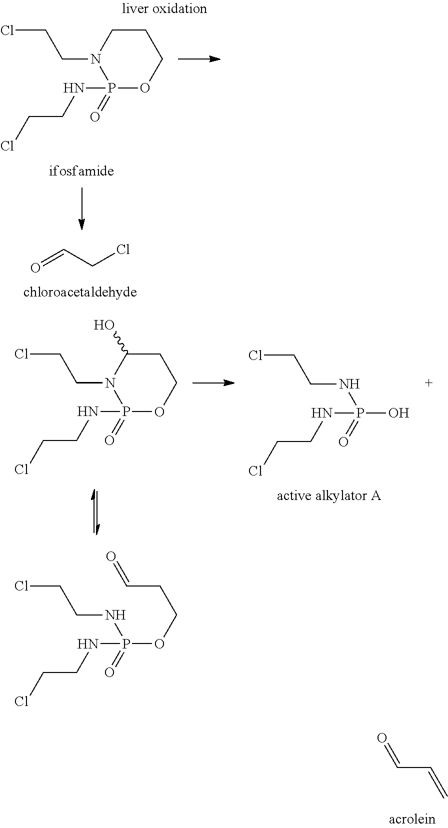

There remains a need for therapy for lymphoma, preferably one that significantly improves the tumor response rate, exhibits a decrease in overall toxicity, and/or allows for more cells to be harvested for transplant. The present invention meets such a need by providing a novel combination therapy as summarized below.

### SUMMARY OF THE INVENTION

The present invention provides methods for treating cancer by administering glufosfamide in combination with anticancer agents. In one aspect the present invention provides a method for treatment of cancer comprising administering to a subject in need thereof a therapeutically effective dose of glufosfamide in combination with carboplatin, etoposide, and optionally rituximab (GCE and RGCE respectively). In particular, the GCE and RGCE therapies have the advantage of decreased toxicity of metabolites, and better side-effect profile. The methods of the present invention require shorter infusion time, permitting out-patient therapy without requiring hospitalization of the patient.

In one embodiment, the present invention provides a method for treating lymphoma. In another embodiment, the present invention provides a method for treating non-Hodgkin's lymphoma. In another embodiment, the present invention provides a method for treating Hodgkin's lymphoma. In another embodiment the treatment is second-line treatment for lymphomas. As discussed above, existing combination therapies have undesirable side-effects and require coadministration of chemoprotective drugs or uroprotectants such as mesna. In contrast, metabolism of glufosfamide does not release the toxic metabolite acrolein. Thus, in one embodiment the present invention does not require coadministration of chemoprotective drugs or uroprotectants such as mesna. In this context, coadministration means administration in the same course of therapy, particularly the same day. In another embodiment, the present invention provides a method of treating cancer comprising administering to a subject in need thereof a therapeutically effective dose of glufosfamide in combination with therapeutically effective doses of carboplatin, etoposide, and optionally rituximab wherein glufosfamide is administered over a period of 1-6 hour. Exemplary therapeutically effective doses are described herein. Therapeutically effective doses of carboplatin and etoposide, for example, are known to physicians.

In one embodiment of the invention, three cycles of GCE (protocol 1) combination chemotherapy are administered at 2-week intervals. Briefly, a 12-hour urine sample is obtained on admission for measurement of the creatinine clearance (Clcr). For example, Etoposide (100 mg/m²) is administered as an intravenous bolus daily for 3 days starting on day 1. Carboplatin is administered as a bolus infusion on day two and dosed at an area under the curve (AUC) of 5, where the dose is calculated using the Calvert formula, 5×(25+Clcr), capped at 800 mg. Glufosfamide (4.5-6.0 g/m²) is administered by infusion for about 1-6 hour beginning on day 2. In another embodiment, glufosfamide (4.5-8.0 g/m²) is administered by infusion for about 1-6 hour beginning on day 2. In another embodiment, glufosfamide is administered at about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² by infusion for about 1-6 hour beginning on day 2. Granulocyte-colony stimulating factor (G-CSF) is optionally administered subcutaneously at 5 μg/kg each day for 8 days (days 5-12).

In one embodiment, 3 cycles of GCE (protocol 2) chemotherapy are administered every 3 weeks as an outpatient treatment. Glufosfamide (1.5-2.0 g/m²) is administered by infusion for 1-6 hours, three consecutive days (days 1, 2 and 3) starting on day 1. In another embodiment, glufosfamide (1.5-3.0 g/m²) is administered by infusion for 1-6 hours, three consecutive days (days 1, 2 and 3) starting on day 1. Etoposide (100 mg/m²) is administered as an intravenous bolus daily for 3 days starting on day 1. Carboplatin is administered as a bolus infusion on day 1, dosed at an area under the curve (AUC) of 5, where the dose is calculated using the Calvert formula, 5×(25+Clcr), capped at 800 mg. Granulocyte-colony stimulating factor (G-CSF) is optionally administered subcutaneously at 5 μg/kg each day from day 5.

In one embodiment, rituximab is administered in addition to the 3 cycles of GCE chemotherapy (protocol 1) as described above. Rituximab (375 mg/m²) is administered on an outpatient basis 48 hours before the initiation of the first cycle of chemotherapy. Following which, rituximab (375 mg/m²) is administered on day 1 of each of the 3 cycles. After the optional administration of oral acetaminophen (650 mg) and intravenous diphenhydramine (50 mg), rituximab is infused according to standard prescribing guidelines. Chemotherapy is administered on an outpatient basis beginning on day 3 of each cycle. A 12-hour urine sample is obtained on admission for measurement of the creatinine clearance (Clcr). Etoposide (100 mg/m²) is administered as an intravenous bolus daily for 3 days, from days 3 to 5. Carboplatin (area under the curve [AUC], 5; dose=5×[25+Clcr]), capped at 800 mg, is administered as a bolus infusion on day 4. Glufosfamide (4.5-6.0 g/m²) is administered for 1-6 hour beginning on day 4. In another embodiment, glufosfamide (4.5-8.0 g/m²) is administered for 1-6 hour beginning on day 4. In another embodiment, glufosfamide is administered at about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² by infusion for about 1-6 hour beginning on day 4. Beginning on day 7, granulocyte-colony stimulating factor (G-CSF) is optionally administered subcutaneously at 5 μg/kg each day for 8 days (days 7-14), after the first 2 cycles of treatment, and at 10 μg/kg per day after the third cycle, until the end of leukopheresis. Cycles are to be administered at 2-week intervals such that the second and third cycles of treatment would begin on day 15 of the previous cycle.

In one embodiment, four doses of rituximab (375 mg/m²) are administered at weekly intervals commencing with cycle 1 of GCE therapy (protocol 2). Three cycles of chemotherapy are administered every 3 weeks as an outpatient treatment. Glufosfamide (1.5-2.0 g/m²) is administered in three equally divided doses by intravenous infusion over 1-6 hours for 3 consecutive days. In another embodiment, glufosfamide (1.5-3.0 g/m²) is administered in three equally divided doses by intravenous infusion over 1-6 hours for 3 consecutive days. Etoposide (100 mg/m²) is administered as an intravenous bolus daily for 3 days. Carboplatin is administered as a bolus infusion on day 1, and dosed at an area under the curve (AUC) of 5, where the dose is calculated using the Calvert formula, 5×(25+Clcr), capped at 800 mg. Granulocyte-colony stimulating factor (G-CSF) is optionally administered subcutaneously at 5 μg/kg each day from day 5.

In another embodiment, glufosfamide is administered in GCE and RGCE therapies as described above by infusion at 1.5-4.0 g/m² once every 3 weeks.

In one embodiment, G-CSF used in the invention is Neupogen®. In another embodiment, G-CSF used in the invention is Neulasta®.

### DETAILED DESCRIPTION OF THE INVENTION

While ICE and RICE combination therapies are used for treating cancer, they exhibit severe toxic side effects in patients. Ifosfamide metabolizes to chloroacetaldehyde and acrolein. In addition to hair loss and nausea/vomiting, ifosfamide metabolite chloroacetaldehyde and acrolein causes central nervous system (CNS) toxicity and bladder irritation that can lead to hemorrhagic cystitis, dysuria, and urinary frequency (see Hardman et al., supra).

CNS effects resulting from ifosfamide-induced neurotoxicity have been observed in 12% of the patients who received ifosfamide. The most common side effects observed are somnolence, confusion, depressive psychosis, and hallucinations. Seizures and coma have also been reported. Approximately 27% of patients treated with ifosfamide experienced encephalopathies, approximately 63% of which were either grade 3 or 4 (Rieger et al., 2004, 15(4):347-345, incorporated herein by reference). In another study (Curtin et al., 1991, Gynecol. Oncol., 42(3):193-6, incorporated herein by reference), 26% of patients experienced grade 4 neurotoxicity.

To ameliorate the bladder irritation caused by ifosfamide, mesna (mercaptoethane sulfonate sodium) is routinely administered concomitantly with ifosfamide. Mesna is an uroprotectant that specifically reduces the hemorrhagic cystitis caused by the ifosfamide metabolite acrolein. However, mesna itself can cause several side effects, including nausea, vomiting, bad taste in the mouth, diarrhea or soft stools, and headache. In addition, up to 6% of patients do not respond to mesna and still go on to develop hematuria.

Given the serious side effects caused by ifosfamide, there is a need for chemotherapy for lymphoma that does not involve ifosfamide. It has been shown recently that the anti-neoplastic active alkylator A can be generated from its glucose conjugate known as glufosfamide (Wiessler et al., U.S. Pat. No. 5,662,936, incorporated herein by reference). In contrast to ifosfamide, metabolism of glufosfamide does not release the toxic metabolite acrolein, and also produces less chloroacetaldehyde (supra).

The present invention arises out of the discovery that using glufosfamide in combination therapies with carboplatin (C), etoposide (E) and Rituximab (R) results in fewer side effects and eliminates the need for co-administration of additional drugs such as mesna that have their own potential side effects. Moreover, glufosfamide can be infused in patients over a shorter infusion period compared to that of ifosfamide. In some administration regimens ifosfamide is infused over a 24 hour period requiring hospitalization of patients.

### Glufosfamide

Glufosfamide also known as α-D-glucosyl-ifosfamide mustard or glc-IPM is a prodrug form of the alkylator A which has been recently used in the clinic to treat cancer (Wiessler et al., supra; Tidmarsh, PCT Patent Publication WO 0576888; Niculescu-Duvaz, 2002, Curr. Opin Investig. Drugs, 3:1527-32; Briasoulis et al., 2000, J. Clin. Oncol., 18(20): 3535-44; Dollner et al., 2004, Anticancer Res. 24(5A):2947-51; and van der Bent et al., 2003, Ann. Oncol., 14(12):1732-4, each of which is incorporated herein by reference). Glufosfamide is hydrolyzed to yield active alkylator A and glucose. In contrast to ifosfamide, glufosfamide metabolism does not produce toxic acrolein and produces less chloroacetaldehyde.

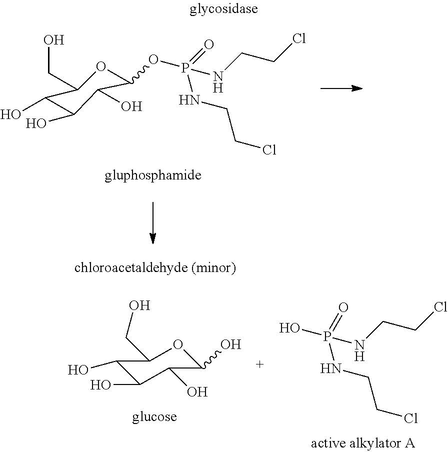

Further, glufosfamide is postulated to enter cells via a sodium dependent cotransporter protein which is upregulated in cancer cells. Such upregulation can lead to cancer cell specific delivery of glufosfamide. In phase II clinical studies, glufosfamide has been administered to patients with pancreatic cancer receiving first line treatment and in patients with non-small cell lung cancer receiving second line chemotherapy, as well as glioblastoma, breast cancer and colon cancer patients (see Niculescu-Duvaz supra). Glufosfamide is routinely administered intravenously; it is contemplated that in the practice of the present invention other administration routes also can be used, such as intrathecal administration, intratumoral injection, oral administration and others. Glufosfamide can be administered at doses comparable to those utilized clinically (see Niculescu-Duvaz supra). In some embodiments, glufosfamide is administered as described elsewhere herein. In various aspects and embodiments of cancer treatment of this invention the anti-neoplastic drugs carboplatin, etoposide, and rituximab are used. Publications cited in this section are intended to illustrate aspects of the drug for the benefit of the practitioner; however, citation to a particular publication in this section or elsewhere in this disclosure is not intended to limit the present invention in any respect, including as to doses, combinations, and indications.

### Subject

A subject is a mammal in need of treatment for cancer. Generally, the subject is a human patient. In some embodiments of the invention, the subject can be a non-human mammal such as a non-human primate, a dog, cat, rabbit, pig, etc. In some embodiments of the invention the subject can be an animal model (e.g., animals such as mice and rats used in screening, characterization and evaluation of medicaments)

### Treatment

As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

For treatment of lymphoma, glufosfamide can be administered to a subject in need of treatment for lymphoma. In certain embodiments, the lymphoma is non-Hodgkin's lymphoma.

### Cancers

While being particularly useful for treatment of lymphomas (including Hodgkin's and non-Hodgkin's lymphoma) the methods of the present invention can be used for treatment of any cancer, including but not limited to breast cancer, pancreatic cancer, cancer of the colon and/or rectum, leukemia, skin cancer, bone cancer, prostate cancer, liver cancer, lung cancer, brain cancer, cancer of the larynx, gallbladder, parathyroid, thyroid, adrenal, neural tissue, head and neck, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, rhabdomyosarcoma, Kaposi's sarcoma, osteogenic and other sarcoma, myeloma, giant cell tumor, small-cell lung tumor, gallstones, islet cell carcinoma, primary brain tumor, acute and chronic lymphocytic and granulocytic tumors, hairy-cell tumor, adenoma, hyperplasia, medullary carcinoma, pheochromocytoma, mucosal neuronms, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, malignant hypercalcemia, renal cell tumor, polycythermia vera, adenocarcinoma, glioblastoma multiforma, leukemias, malignant melanomas, and epidermoid carcinomas. In one embodiment, the present invention provides a method for treating lymphomas. In another embodiment, the present invention provides a method for treating non-Hodgkin's lymphoma.

### Administration Regimens

It will be appreciated that cancer treatment by chemotherapy sometimes involves multiple “rounds” or “cycles” of administration of a drug, where each cycle comprises administration of the drug one or more times according to a specified schedule (e.g., daily; once per week; multiple times a week either on consecutive days or non-consecutive days; once every cycle; multiple times every cycle for example every three weeks for three consecutive days; etc., wherein each cycle range from 1 week up to several weeks, preferably 2, 3, 4, 5, 6, 7, or 8 weeks). For example, chemotherapeutic drugs can be administered for from 1 to 8 cycles, or for a longer period. When more than one drug (e.g., two drugs) is administered to a subject, each can be administered according to its own schedule as illustrated above (e.g., weekly; once every three weeks; etc.). It will be clear that administration of drugs, even those administered with different periodicity, can be coordinated so that both drugs are administered on the same day at least some of the time or, alternatively, so the drugs are administered on consecutive days at least some of the time.

As is understood in the art, treatment with cancer therapeutic drugs can be suspended temporarily if toxicity is observed, or for the convenience of the patient, without departing from the scope of the invention, and then resumed.

### Administration in Combination

During chemotherapy treatment of cancer two, three, or four drugs can be administered to a subject “in combination” by administering them as part of the same course of therapy. A course of therapy refers to administration of combinations of drugs believed by the medical professional to work together additively, complementarily, synergistically, or otherwise to produce a more favorable outcome than that anticipated for administration of a single drug. A course of therapy can be for one or a few days, but more often extends for several weeks.

When two or more drugs are administered in combination, a variety of schedules can be used. In one case, for example and without limitation, Drug 1 is first administered prior to administration of Drug 2, and treatment with Drug 1 is continued throughout the course of administration of Drug 2; alternatively Drug 1 is administered after the initiation or completion of Drug 2 therapy; alternatively, Drug 1 is first administered contemporaneously with the initiation of the other cancer therapy. As used in this context, “contemporaneously” means the two drugs are administered the same day, or on consecutive days.

Although in principle certain drugs can be co-formulated, in general they are administered in separate compositions. Similarly, although certain drugs can be administered simultaneously, more often (especially for drugs administered by infusion) drugs are administered at different times on the same day, on consecutive days, or according to another schedule.

### GCE and RGCE

In one aspect the present invention provides a method for the treatment of cancer comprising administering to a subject in need thereof a therapeutically effective dose of glufosfamide in combination with carboplatin, etoposide, and optionally rituximab (GCE and RGCE respectively). In one embodiment the cancer is lymphoma. In another embodiment, the cancer is non-Hodgkin's lymphoma. In another embodiment, the cancer is selected from Hodgkin's lymphomas. In another embodiment the treatment is second-line treatment for lymphomas. A variety of cancers can be treated by this method as described herein in detail.

As discussed above, existing combination therapies including ifosfamide have undesirable side-effects and require coadministration of chemoprotective drugs and/or uroprotectants such as mesna. In contrast, metabolism of glufosfamide does not produce the toxic metabolite acrolein and produces in vivo less chloroacetaldehyde than ifosfamide. In another embodiment the present invention does not require coadministration of chemoprotective drugs or uroprotectants such as mesna. In another embodiment, the present invention provides a method of treating cancer comprising administering to a subject in need thereof a therapeutically effective dose of glufosfamide in combination with carboplatin, etoposide, and optionally rituximab wherein glufosfamide is administered over a period of 1-6 hour. Glufosfamide can be administered over a shorter time period compared to ifosfamide which requires a 24 hour infusion in ICE and RICE therapies.

Glufosfamide, in combination with carboplatin and etoposide or carboplatin, etoposide, and rituximab, as used in the present invention can be administered at any dose that is therapeutically effective, such as doses comparable to those routinely utilized clinically. Specific dose regimens for known and approved antineoplastic agents (e.g., the recommended effective dose) are known to physicians and are given, for example, in the product descriptions found in the references Oradell, N J, Physicians' Desk Reference, 2003, 57th Ed., Medical Economics Company, Inc.; Hardman et al., supra; and/or are available from the Federal Drug Administration; and/or are discussed in the medical literature.

In one embodiment, glufosfamide is administered for 1, 2, 3, 4, 5, 6, 7, 8, or more than 8 cycles, each cycle comprising infusion in the range of:

a) about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² or over an infusion period of 1-6 hours once every three weeks;

b) about 1.5 to about 3.0 g/m² or about 1.5 to about 2.0 g/m² over an infusion period of 1-6 hours for three consecutive days (days 1, 2 and 3) every three weeks;

c) about 1.5 to about 2.0 g/m² over an infusion period of 1-6 hours once per week; or

d) about 1.5 to about 8.0 g/m²; about 1.5 to about 6.0 g/m²; or about 1.5 to about 4.5 g/m² over an infusion period of 1-6 hours once every four weeks.

As used in this context an “infusion period of 1-6 hours” includes an infusion period of about 1, about 2, about 3, about 4, about 5 and about 6 hours.

In another embodiment, glufosfamide is administered on weeks 1, 2, 3, 5, 6 and 7 of a seven-week cycle, and the administration is for 1, 2, 3, 4, or more than 4 seven-week cycles, where each cycle comprises infusion of:

a) about 1.0 g/m² over a period of about 30 min;

b) about 2.2 g/m² over a period of about 30 min;

c) about 1.5 g/m² over a period of about 150 min.

In one embodiment, carboplatin is administered for 1, 2, 3, 4, 5, 6, 7, 8, or more than 8 cycles, each cycle comprising a bolus infused over about 10 to about 60 minutes and dosed at an area under the curve (AUC) of 5, where the dose is calculated using the Calvert formula, 5×(25+Clcr), capped at 800 mg.

In another embodiment, carboplatin is administered on weeks 1, 2, 3, 5, 6 and 7 of a seven-week cycle, and the administration is for 1, 2, 3, 4, or more than 4 seven-week cycles, where each cycle comprises a bolus administered over 15, 30, or 60 minutes and dosed at an area under the curve (AUC) of 5, where the dose is calculated using the Calvert formula, 5×(25+Clcr), capped at 800 mg;

In one embodiment, Etoposide is administered for 1, 2, 3, 4, 5, 6, 7, 8, or more than 8 cycles, each cycle comprising administering in the range of:

a) about 50-120 mg/m² as an intravenous bolus infused over 30-60 minutes for three days every three or four weeks;

b) about 30-75 mg/m² orally for twenty one days; or

c) about 30-75 mg/m² as an intravenous bolus once every week.

In one embodiment, rituximab is administered for 1, 2, 3, 4, 5, 6, 7, 8 or more than 8 cycles, each cycle comprising infusion in the range of about 350-400, 350-390, and 360-375 mg/m² rituximab once every 3-4 weeks;

In another embodiment, rituximab is administered on weeks 1, 2, 3, 5, 6 and 7 of a seven-week cycle, and the administration is for 1, 2, 3, 4, or more than 4 seven-week cycles, where each cycle comprises infusion of about 350, 375, or 400 mg/m² rituximab;

In one embodiment of the invention, three cycles of GCE (protocol 1) combination chemotherapy are administered at 2-week intervals. Briefly, a 12-hour urine sample is obtained on admission for measurement of the creatinine clearance (Clcr). Etoposide (100 mg/m²) is administered as an intravenous bolus daily for 3 days starting on day 1. Carboplatin is administered as a bolus infusion on day two and dosed at an area under the curve (AUC) of 5, where the dose is calculated using the Calvert formula, 5×(25+Clcr), capped at 800 mg. Glufosfamide (4.5-6.0 g/m²) is administered by infusion for about 1-6 hour beginning on day 2. In another embodiment, glufosfamide (4.5-8.0 g/m²) is administered by infusion for about 1-6 hour beginning on day 2. Granulocyte-colony stimulating factor (G-CSF) is optionally administered subcutaneously at 5 μg/kg each day for 8 days (days 5-12).

In one embodiment, 3 cycles of GCE (protocol 2) chemotherapy are administered every 3 weeks as an outpatient treatment. Glufosfamide (1.5-2.0 g/m²) is administered by infusion for 1-6 hours, three consecutive days (days 1, 2 and 3) starting on day 1. In another embodiment, glufosfamide (1.5-3.0 g/m²) is administered by infusion for 1-6 hours, three consecutive days (days 1, 2 and 3) starting on day 1. Etoposide (100 mg/m²) is administered as an intravenous bolus daily for 3 days starting on day 1. Carboplatin is administered as a bolus infusion on day 1, dosed at an area under the curve (AUC) of 5, where the dose is calculated using the Calvert formula, 5×(25+Clcr), capped at 800 mg. Granulocyte-colony stimulating factor (G-CSF) is optionally administered subcutaneously at 5 μg/kg each day from day 5.

In one embodiment, rituximab is administered in addition to the 3 cycles of GCE chemotherapy (protocol 1) as described above. Rituximab (375 mg/m²) is administered on an outpatient basis 48 hours before the initiation of the first cycle of chemotherapy. Following which, rituximab (375 mg/m²) is administered on day 1 of each of the 3 cycles. After the optional administration of oral acetaminophen (650 mg) and intravenous diphenhydramine (50 mg), rituximab is infused according to standard prescribing guidelines. Chemotherapy is administered on an outpatient basis beginning on day 3 of each cycle. A 12-hour urine sample is obtained on admission for measurement of the creatinine clearance (Clcr). Etoposide (100 mg/m²) is administered as an intravenous bolus daily for 3 days, from days 3 to 5. Carboplatin (area under the curve [AUC], 5; dose=5×[25+Clcr]), capped at 800 mg, is administered as a bolus infusion on day 4. Glufosfamide (4.5-6.0 g/m²) is administered for 1-6 hour beginning on day 4. In another embodiment, glufosfamide (4.5-8.0 g/m²) is administered for 1-6 hour beginning on day 4. Beginning on day 7, granulocyte-colony stimulating factor (G-CSF) is optionally administered subcutaneously at 5 μg/kg each day for 8 days (days 7-14), after the first 2 cycles of treatment, and at 10 μg/kg per day after the third cycle, until the end of leukopheresis. Cycles are to be administered at 2-week intervals such that the second and third cycles of treatment would begin on day 15 of the previous cycle.

In one embodiment, four doses of rituximab (375 mg/m²) are administered at weekly intervals commencing with cycle 1 of GCE therapy (protocol 2). Three cycles of chemotherapy are administered every 3 weeks as an outpatient treatment. Glufosfamide (1.5-2.0 g/m²) is administered in three equally divided doses by intravenous infusion over 1-6 hours for 3 consecutive days. In another embodiment, glufosfamide (1.5-3.0 g/m²) is administered in three equally divided doses by intravenous infusion over 1-6 hours for 3 consecutive days. Etoposide (100 mg/m²) is administered as an intravenous bolus daily for 3 days. Carboplatin is administered as a bolus infusion on day 1, and dosed at an area under the curve (AUC) of 5, where the dose is calculated using the Calvert formula, 5×(25+Clcr), capped at 800 mg. Granulocyte-colony stimulating factor (G-CSF) is optionally administered subcutaneously at 5 μg/kg each day from day 5.

In another embodiment, glufosfamide is administered in GCE and RGCE therapies as described above by infusion at 1.5-4.0 g/m² once every 3 weeks.

In one embodiment, G-CSF used in the invention is Neupogen®. In another embodiment, G-CSF used in the invention is Neulasta®.

In one embodiment, the present invention provides a method for treatment of cancer comprising administering to a subject in need thereof a therapeutically effective dose of glufosfamide in combination with 2-deoxyglucose, carboplatin, etoposide, and optionally rituximab. In another embodiment, other anti-neoplastic agents can be used in combination with GCE or RGCE therapy for treatment of cancer.

The present invention having been described in detail in the preceding sections, the following examples are provided to illustrate certain aspects of, but not to limit, the invention.

### Example

### Cell Lines and Reagents

Ramos cells lines (Burkitt's lymphoma) were obtained from the American Type Culture Collection (ATCC, Rockville, Md.). All cell lines were cultured in Dulbecco's modified Eagle's medium supplemented with 10% fetal calf serum, sodium pyruvate, nonessential amino acids, L-glutamine, vitamins, and antibiotics. Cells were maintained in a humidified incubator containing 5% CO₂, 21% O₂, at 37° C. All chemical reagents were purchased from Sigma Chemical Co. (St. Louis, Mo.) unless otherwise specified. Glufosfamide was provided directly by Threshold Pharmaceuticals and reconstituted in PBS freshly for each study.

### Cell Proliferation Assay

To determine the effect of the combinations of the present invention on cell proliferation, the antiproliferative activity of these combinations was tested in a multi-well Alamar Blue based assay (at 3 days). Cell growth in the presence and absence of the test compound as tabulated in Table 1 was compared, as measured by a fluorescence plate reader at excitation 550 nm and emission 590 nm (see Biosource International Inc., Tech Application Notes, Use of Alainar Blue in the measurement of Cell Viability and Toxicity, Determining IC₅₀). Ramos cells (Burkitt's lymphoma) (ATCC Catalog #CRL-1596), 3000 cells/well/180 μl) were seeded in a 96 well plate in RPMI medium (Invitrogen Corporation, Carlsbad, Calif.) and incubated overnight. After 24 hours, these plates were divided into Control group and 3 day treatment group.

A test compound (Glufosfamide, Carboplatin, or Etoposide) was added to each plate in the treatment groups at a concentration as tabulated in Tables 1A-1D (in 200 μl of medium, respectively). The cells were incubated for 3 days, followed by staining with AlamarBlue. In the Control group, AlamarBlue was added to the plate at (i) day 0 and (ii) day 3 and measured to establish the control reading. In all the groups, the capacity of the cells to proliferate was measured 17 hours after addition of AlamarBlue using a fluorescence plate reader at excitation 550 nm and emission 590 nm. The results of the assay are tabulated in Tables 1A-D. Dose response curves for Glufosfamide, Carboplatin and Etoposide were generated, and the IC₁₀, 2×IC₁₀, 0.5×IC₁₀ and 0.25×IC₁₀ values for Carboplatin and Etoposide were determined. Dose dependent percent inhibition was calculated using Graphpad Prism® (GraphPad Software, Inc.). These values were then applied against the Combination Index (CI) method as described by Chou and Talalay (Chou, T. C. and Talalay, P., Quantitative analysis of dose-effect relationships: The combined effects of multiple drugs or enzyme inhibitors. Adv. Enz. Regul. 22:27-55, 1984) using CalcuSyn software (Biosoft).

TABLE 1A

| | Cell | | Cell | | Cell |
|---|---|---|---|---|---|
| Glufosfamide | proliferation | Carboplatin | proliferation | Etoposide | proliferation |
| (μM) | as % control | (μM) | as % control | (μM) | as % control |
| 0 (Control) | 100 | 0 (Control) | 100 | 0 (Control) | 100 |
| 0.7 | 91 | 0.4 | 97 | 0.13 | 37 |
| 2 | 92 | 1.2 | 98 | 0.4 | 26 |
| 6 | 94 | 3.7 | 94 | 1.2 | 27 |
| 19 | 81 | 11 | 68 | 3.7 | 3 |
| 56 | 65 | 33 | 46 | 11 | 0 |
| 167 | 65 | 100 | 0 | 33 | 0 |
| 500 | 0 | 300 | 0 | 100 | 0 |

TABLE 1B

| Compound (single agent) | IC₅₀ (μM) |
|---|---|
| Glufosfamide (G) | 180 |
| Carboplatin (C) | 25 |
| Etoposide (E) | <0.13 |

TABLE 1C

| | Cell | | Cell | | Cell |
|---|---|---|---|---|---|
| Glufosfamide | proliferation | Carboplatin | proliferation | Etoposide | proliferation |
| (μM) | as % control | (μM) | as % control | (μM) | as % control |
| 0 (control) | 100 | 0 (Control) | 100 | 0 (Control) | 100 |
| — | — | — | — | 0.004 | 97 |
| 9 | 91 | 0.4 | 98 | 0.013 | 91 |
| 19 | 79 | 1.2 | 98 | 0.04 | 62 |
| 37.5 | 70 | 3.7 | 89 | 0.12 | 28 |
| 75 | 64 | 11 | 74 | 0.37 | 35 |
| 150 | 54 | 33 | 45 | 1.1 | 14 |
| 300 | 11 | 100 | 0 | 3.3 | 0 |
| 600 | 0 | 300 | 0 | 10 | 0 |

TABLE 1D

| Compound (single agent) | IC₅₀ (μM) |
|---|---|
| Glufosfamide (G) | 120 |
| Carboplatin (C) | 25 |
| Etoposide (E) | 0.076 |

The effect of the combinations of the present invention on cell proliferation was determined by comparing cell growth in the presence and absence of the test combinations as tabulated in the tables below according to the procedure described above. Glufosfamide was added to combination groups (Group A−0.75 μM C+0.0025 μM E; Group B−1.5 μM C+0.005 μM E; Group C−3 C+0.01 μM E; and Group D−6 μM C+0.02 μM E) at a concentration as tabulated in Tables 2A-2D and the IC50 of the GCE combinations were calculated and tabulated in Table 2E.

TABLE 2A
0.75 μM C + 0.0025 μM E

| G (μM) | Cell proliferation as % control |
|---|---|
| 0 (control) | 97 |
| 9 | 83 |
| 19 | 78 |
| 37.5 | 71 |
| 75 | 64 |
| 150 | 55 |
| 300 | 11 |
| 600 | 0 |

TABLE 2B
1.5 μM C + 0.005 μM E

| G (μM) | Cell proliferation as % control |
|---|---|
| 0 (control) | 88 |
| 9 | 79 |
| 19 | 77 |
| 37.5 | 71 |
| 75 | 63 |
| 150 | 42 |
| 300 | 11 |
| 600 | 0 |

TABLE 2C
3 μM C + 0.01 μM E

| G (μM) | Cell proliferation as % control |
|---|---|
| 0 (control) | 86 |
| 9 | 74 |
| 19 | 74 |
| 37.5 | 62 |
| 75 | 55 |
| 150 | 47 |
| 300 | 9 |
| 600 | 0 |

TABLE 2D
6 μM C + 0.02 μM E

| G (μM) | Cell proliferation as % control |
|---|---|
| 0 (control) | 66 |
| 9 | 62 |
| 19 | 59 |
| 37.5 | 59 |
| 75 | 57 |
| 150 | 51 |
| 300 | 9 |
| 600 | 0 |

TABLE 2E

| Combination | IC₅₀ (μM) |
|---|---|
| Glufosfamide + 0.75 μM C + 0.0025 μM E | 120 |
| Glufosfamide + 1.5 μM C + 0.005 μM E | 100 |
| Glufosfamide + 3 μM C + 0.01 μM E | 80 |
| Glufosfamide + 6 μM C + 0.02 μM E | 80 |

### EQUIVALENTS AND INCORPORATION BY REFERENCE

While the present invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications and patent documents cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

1. A method for treatment of cancer comprising administering to a subject in need thereof a therapeutically effective dose of glufosfamide in combination with carboplatin and etoposide.

2. The method of claim 1 further comprising administering rituximab.

3. The method of claim 1 wherein said cancer is a lymphoma.

4. The method of claim 3 wherein said cancer is a non-Hodgkin's lymphoma.

5. The method of claim 3 wherein said cancer is a Hodgkin's lymphoma

6. The method of claim 1 wherein said glufosfamide is administered in the absence of mesna.

7. The method of claim 1 wherein glufosfamide is administered for one or more dosage cycles, each cycle comprising infusion in the range of:
a): about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² or over an infusion period of 1-6 hours once every three weeks;
b): about 1.5 to about 3.0 g/m² or about 1.5 to about 2.0 g/m² over an infusion period of 1-6 hours for three consecutive days (days 1, 2 and 3): every three weeks;
c): about 1.5 to about 2.0 g/m² over an infusion period of 1-6 hours once per week; or
d): about 1.5 to about 8.0 g/m²; about 1.5 to about 6.0 g/m²; or about 1.5 to about 4.5 g/m² over an infusion period of 1-6 hours once every four weeks.

8. The method of claim 2 wherein said cancer is a lymphoma.

9. The method of claim 8 wherein said cancer is a non-Hodgkin's lymphoma.

10. The method of claim 8 wherein said cancer is a Hodgkin's lymphoma

11. The method of claim 2 wherein said glufosfamide is administered in the absence of mesna.

12. The method of claim 8 wherein said glufosfamide is administered in the absence of mesna.

13. The method of claim 2 wherein glufosfamide is administered for one or more dosage cycles, each cycle comprising infusion in the range of:
a): about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² or over an infusion period of 1-6 hours once every three weeks;
b): about 1.5 to about 3.0 g/m² or about 1.5 to about 2.0 g/m² over an infusion period of 1-6 hours for three consecutive days (days 1, 2 and 3): every three weeks;
c): about 1.5 to about 2.0 g/m² over an infusion period of 1-6 hours once per week; or
d): about 1.5 to about 8.0 g/m²; about 1.5 to about 6.0 g/m²; or about 1.5 to about 4.5 g/m² over an infusion period of 1-6 hours once every four weeks.

14. The method of claim 3 wherein glufosfamide is administered for one or more dosage cycles, each cycle comprising infusion in the range of:
a): about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² or over an infusion period of 1-6 hours once every three weeks;
b): about 1.5 to about 3.0 g/m² or about 1.5 to about 2.0 g/m² over an infusion period of 1-6 hours for three consecutive days (days 1, 2 and 3): every three weeks;
c): about 1.5 to about 2.0 g/m² over an infusion period of 1-6 hours once per week; or
d): about 1.5 to about 8.0 g/m²; about 1.5 to about 6.0 g/m²; or about 1.5 to about 4.5 g/m² over an infusion period of 1-6 hours once every four weeks.

15. The method of claim 4 wherein glufosfamide is administered for one or more dosage cycles, each cycle comprising infusion in the range of:
a): about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² or over an infusion period of 1-6 hours once every three weeks;
b): about 1.5 to about 3.0 g/m² or about 1.5 to about 2.0 g/m² over an infusion period of 1-6 hours for three consecutive days (days 1, 2 and 3): every three weeks;
c): about 1.5 to about 2.0 g/m² over an infusion period of 1-6 hours once per week; or
d): about 1.5 to about 8.0 g/m²; about 1.5 to about 6.0 g/m²; or about 1.5 to about 4.5 g/m² over an infusion period of 1-6 hours once every four weeks.

16. The method of claim 5 wherein glufosfamide is administered for one or more dosage cycles, each cycle comprising infusion in the range of:
a): about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² or over an infusion period of 1-6 hours once every three weeks;
b): about 1.5 to about 3.0 g/m² or about 1.5 to about 2.0 g/m² over an infusion period of 1-6 hours for three consecutive days (days 1, 2 and 3): every three weeks;
c): about 1.5 to about 2.0 g/m² over an infusion period of 1-6 hours once per week; or
d): about 1.5 to about 8.0 g/m²; about 1.5 to about 6.0 g/m²; or about 1.5 to about 4.5 g/m² over an infusion period of 1-6 hours once every four weeks.

17. The method of claim 6 wherein glufosfamide is administered for one or more dosage cycles, each cycle comprising infusion in the range of:
a): about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² or over an infusion period of 1-6 hours once every three weeks;
b): about 1.5 to about 3.0 g/m² or about 1.5 to about 2.0 g/m² over an infusion period of 1-6 hours for three consecutive days (days 1, 2 and 3): every three weeks;
c): about 1.5 to about 2.0 g/m² over an infusion period of 1-6 hours once per week; or
d): about 1.5 to about 8.0 g/m²; about 1.5 to about 6.0 g/m²; or about 1.5 to about 4.5 g/m² over an infusion period of 1-6 hours once every four weeks.

18. The method of claim 8 wherein glufosfamide is administered for one or more dosage cycles, each cycle comprising infusion in the range of:
a): about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² or over an infusion period of 1-6 hours once every three weeks;
b): about 1.5 to about 3.0 g/m² or about 1.5 to about 2.0 g/m² over an infusion period of 1-6 hours for three consecutive days (days 1, 2 and 3): every three weeks;
c): about 1.5 to about 2.0 g/m² over an infusion period of 1-6 hours once per week; or
d): about 1.5 to about 8.0 g/m²; about 1.5 to about 6.0 g/m²; or about 1.5 to about 4.5 g/m² over an infusion period of 1-6 hours once every four weeks.

19. The method of claim 9 wherein glufosfamide is administered for one or more dosage cycles, each cycle comprising infusion in the range of:
a): about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² or over an infusion period of 1-6 hours once every three weeks;
b): about 1.5 to about 3.0 g/m² or about 1.5 to about 2.0 g/m² over an infusion period of 1-6 hours for three consecutive days (days 1, 2 and 3): every three weeks;
c): about 1.5 to about 2.0 g/m² over an infusion period of 1-6 hours once per week; or
d): about 1.5 to about 8.0 g/m²; about 1.5 to about 6.0 g/m²; or about 1.5 to about 4.5 g/m² over an infusion period of 1-6 hours once every four weeks.

20. The method of claim 10 wherein glufosfamide is administered for one or more dosage cycles, each cycle comprising infusion in the range of:
a): about 1.5 to about 8.0 g/m²; about 1.5 to about 6.0 g/m²; about 1.5 to about 4.5 g/m²; about 4.5 to about 8.0 g/m²; about 4.5 to about 6.0 g/m²; or about 4.5 to about 5.0 g/m² or over an infusion period of 1-6 hours once every three weeks;
b): about 1.5 to about 3.0 g/m² or about 1.5 to about 2.0 g/m² over an infusion period of 1-6 hours for three consecutive days (days 1, 2 and 3): every three weeks;
c): about 1.5 to about 2.0 g/m² over an infusion period of 1-6 hours once per week; or
d): about 1.5 to about 8.0 g/m²; about 1.5 to about 6.0 g/m²; or about 1.5 to about 4.5 g/m² over an infusion period of 1-6 hours once every four weeks.
